(19) United States
(12) Patent Application Publication
(10) Pub. No.: US 2012/0020907 A1
(43) Pub. Date: Jan. 26, 2012

(54) LONG-WEAR MASCARA COMPOSITION

(75) Inventors: Hy Si Bui, Piscataway, NJ (US); Mohamed Kanji, Edison, NJ (US); Chunhua Li, Scotch Plains, NJ (US); Bruno Thierry Bavouzet, Gentilly (FR)

(73) Assignee: L'Oreal S.A., Paris (FR)

(21) Appl. No.: 13/172,424

(22) Filed: Jun. 29, 2011

Related U.S. Application Data
(60) Provisional application No. 61/359,743, filed on Jun. 29, 2010.
(60) Provisional application No. 61/359,724, filed on Jun. 29, 2010.
(60) Provisional application No. 61/359,704, filed on Jun. 29, 2010.

(51) Int. Cl. A61K 8/81; A61Q 5/06

(52) U.S. Cl. 424/70.7

(57) ABSTRACT

The present invention is directed to long wearing, water-resistant mascara including an oil-soluble polar modified polymer and an amine compound.

### CROSS-REFERENCE TO RELATED APPLICATIONS

This application claims the benefit of priority under 35 U.S.C. §119(e) from U.S. Provisional Application Ser. Nos. 61/359,704; 61/359,724; and 61/359,743, all filed Jun. 29, 2010, the entire contents of which are hereby incorporated by reference.

### FIELD OF THE INVENTION

The present invention generally relates to a novel mascara composition and method of making-up eye lashes. More particularly, the present invention relates to a mascara composition having excellent shine, water-proof and/or smudge-resistance properties, having improved properties relating to removability from their containers, removability after application using water and/or volumization.

### BACKGROUND OF THE INVENTION

It has been known that mascaras are usually structured with waxes. The conventional waxes used in mascara formulations have crystals that are large enough to diffract/scatter light, giving the formula an opaque and dull appearance.

It is also well known in the industry that one way of formulating a mascara composition which has long wear properties is to make it either anhydrous or include latex film formers based on oil-in-water emulsions.

Both techniques have numerous drawbacks. First, they are somewhat expensive. Second, they can be difficult to formulate with due to the large solid content load required, making them unstable, or sensitive to added ingredients.

Therefore, it is desirable to provide an eye makeup composition which provides excellent shine, color deposition, and long-wear properties.

### BRIEF SUMMARY OF THE INVENTION

A first aspect of the present invention is directed to long wearing, water-resistant, color enhanced eye makeup compositions. The compositions comprise: (a) at least one oil soluble polar modified polymer; and (b) at least one amine compound selected from the group consisting of a basic amino acid and a vinylamine/vinylamide copolymer.

The present invention also relates to compositions comprising: (a) at least one oil soluble polar modified polymer; b) at least one amine compound selected from the group consisting of a basic amino acid and a vinylamine/vinylamide copolymer; and (c) water.

The present invention also relates to compositions comprising: (a) at least one oil soluble polar modified polymer; b) at least one amine compound selected from the group consisting of a basic amino acid and a vinylamine/vinylamide copolymer; (c) water; and (d) at least one aqueous polyurethane dispersion.

The present invention also relates to long wearing, water-resistant, color enhanced eye makeup compositions which can be removed with water. The compositions include (a) a reaction product of at least one basic amino acid and at least one oil-soluble polar modified polymer; and (b) at least one aqueous polyurethane dispersion.

The present invention also relates to a mascara composition made by combining: (a) at least one basic amino acid; (b) at least one aqueous polyurethane dispersion; (c) at least one oil-soluble polar modified polymer; and (d) water.

The present invention also relates to water-resistant, color enhanced eye makeup compositions. The compositions include a reaction product of at least one amine compound selected from the group consisting of a basic amino acid and a vinylamine/vinylamide copolymer, and at least one oil-soluble polar modified polymer.

The present invention also relates to a mascara composition made by combining: (a) at least one amine compound selected from the group consisting of a basic amino acid and a vinylamine/vinylamide copolymer; and (b) at least one oil-soluble polar modified polymer.

Another aspect of the present invention is directed to a method of making up eyelashes involving applying the above-disclosed compositions to eyelashes.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

“Film former” or “film forming agent” or “film forming resin” as used herein means a polymer which, after dissolution in at least one solvent (such as, for example, water and organic solvents), leaves a film on the substrate to which it is applied, for example, once the at least one solvent evaporates, absorbs and/or dissipates on the substrate.

“Keratinous substrates”, as used herein, include but are not limited to, skin, hair and nails.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, weeks, 4 weeks and 8 weeks. At each inspection, the sample is examined for abnormalities in the composition such as phase separation if the composition is in the form of an emulsion, bending or leaning if the composition is in stick form, melting, or syneresis (or sweating). The stability is further tested by repeating the 8-week test at 37° C., 40° C., 45° C., 50° C., and under freeze-thaw conditions. A composition is considered to lack stability if in any of these tests an abnormality that impedes functioning of the composition is observed. The skilled artisan will readily recognize an abnormality that impedes functioning of a composition based on the intended application.

“Volatile”, as used herein, means having a flash point of less than about 100° C. “Non-volatile”, as used herein, means having a flash point of greater than about 100° C.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc. In a preferred embodiment of the present invention, little or no composition is transferred from the wearer.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

Oil-Soluble Polar Modified Polymer

According to the present invention, compositions comprising at least one oil-soluble polar modified polymer are provided. “Polar modified polymer” as used herein refers to a hydrophobic homopolymer or copolymer which has been modified with hydrophilic unit(s). “Oil-soluble” as used herein means that the polar modified polymer is soluble in oil.

Suitable monomers for the hydrophobic homopolymers and/or copolymers include, but are not limited to, cyclic, linear or branched, substituted or unsubstituted, C2-C20 compounds such as, for example, styrene, ethylene, propylene, isopropylene, butylene, isobutylene, pentene, isopentene, isoprene, hexene, isohexene, decene, isodecene, and octadecene, including all ranges and subranges therebetween. Preferably, the monomers are C2-C8 compounds, more preferably C2-C6 compounds, and most preferably C2-C4 compounds such as ethylene, propylene and butylene.

Suitable hydrophilic unit(s) include, but are not limited to, maleic anhydride, acrylates, alkyl acrylates such as, for example, methyl acrylate, ethyl acrylate, propyl acrylate, and butyl acrylate, and polyvinylpyrrolidone (PVP).

According to the present invention, the polar modified polymer is oil-soluble: that is, the polymer does not contain a sufficient amount of hydrophilic unit(s) to render the entire polymer water-soluble or oil-insoluble. According to preferred embodiments, the polar modified polymer contains the same amount of hydrophobic monomer as hydrophilic unit (1:1 ratio) or more hydrophobic monomer than hydrophilic unit. According to particularly preferred embodiments, the polar modified polymer contains 50% or less hydrophilic unit(s) (based on weight of the polymer), 40% or less hydrophilic unit(s), 30% or less hydrophilic unit(s), 20% or less hydrophilic unit(s), 10% or less hydrophilic unit(s), 5% or less hydrophilic unit(s), 4% or less hydrophilic unit(s), or 3% or less hydrophilic unit(s).

Preferably, the polar modified polymer has from about 0.5% to about 10% hydrophilic units, more preferably from about 1% to about 8% hydrophilic units by weight with respect to the weight of the polymer, including all ranges and subranges therebetween. Particularly preferred hydrophilically modified polymers are ethylene and/or propylene homopolymers and copolymers which have been modified with maleic anhydride units.

According to preferred embodiments of the present invention, the polar modified polymer is a wax. According to particularly preferred embodiments, the polar modified wax is made via metallocene catalysis, and includes polar groups or units as well as a hydrophobic backbone. Suitable modified waxes include those disclosed in U.S. patent application publication no. 20070031361, the entire contents of which is hereby incorporated by reference. Particularly preferred polar modified waxes are C2-C3 polar modified waxes.

In accordance with preferred embodiments of the present invention, the polar modified wax is based upon a homopolymer and/or copolymer wax of hydrophobic monomers and has a weight-average molecular weight Mw of less than or equal to 25 000 g/mol, preferably of 1000 to 22 000 g/mol and particularly preferably of 4000 to 20,000 g/mol, a number-average molecular weight Mn of less than or equal to 15 000 g/mol, preferably of 500 to 12 000 g/mol and particularly preferably of 1000 to 5000 g/mol, a molar mass distribution Mw/Mn in the range from 1.5 to 10, preferably from 1.5 to 5, particularly preferably from 1.5 to 3 and especially preferably from 2 to 2.5, which have been obtained by metallocene catalysis. Also, the polar modified wax preferably has a melting point above 75° C., more preferably above 90° C. such as, for example, a melting point between 90° C. and 160° C., preferably between 100° C. and 150° C., including all ranges and subranges therebetween.

In the case of a copolymer wax, it is preferable to have, based on the total weight of the copolymer backbone, 0.1 to 30% by weight of structural units originating from the one monomer and 70.0 to 99.9% by weight of structural units originating from the other monomer. Such homopolymer and copolymer waxes can be made, for example, by the process described in EP 571 882, the entire contents of which is hereby incorporated by reference, using the metallocene catalysts specified therein. Suitable preparation processes include, for example, suspension polymerization, solution polymerization and gas-phase polymerization of olefins in the presence of metallocene catalysts, with polymerization in the monomers also being possible.

Polar modified waxes can be produced in a known manner from the hompopolymers and copolymers described above by oxidation with oxygen-containing gases, for example air, or by graft reaction with polar monomers, for example maleic acid or acrylic acid or derivatives of these acids. The polar modification of metallocene polyolefin waxes by oxidation with air is described, for example, in EP 0 890 583 A1, and the modification by grafting is described, for example, in U.S. Pat. No. 5,998,547, the entire contents of both of which are hereby incorporated by reference in their entirety.

Acceptable polar modified waxes include, but are not limited to, homopolymers and/or copolymers of ethylene and/or propylene groups which have been modified with hydrophilic units such as, for example, maleic anhydride, acrylate, methacrylate, polyvinylpyrrolidone (PVP), etc. Preferably, the C2-C3 wax has from about 0.5% to about 10% hydrophilic units, more preferably from about 1% to about 8% hydrophilic units by weight with respect to the weight of the wax, including all ranges and subranges therebetween. Particularly preferred hydrophilically modified waxes are ethylene and/or propylene homopolymers and copolymers which have been modified with maleic anhydride units.

Particularly preferred C2-C3 polar modified waxes for use in the present invention are polypropylene and/or polyethylene-maleic anhydride modified waxes (“PEMA,” “PPMA.” “PEPPMA”) commercially available from Clariant under the trade name LICOCARE or LICOCENE, Specific examples of such waxes include products marketed by Clariant under the LicoCare name having designations such as PP207.

Other suitable polar modified polymers include, but are not limited to A-C 573 A (ETHYLENE-MALEIC ANHYDRIDE COPOLYMER; Drop Point, Mettler: 106° C.) from Honeywell, A-C 596 A (PROPYLENE-MALEIC ANHYDRIDE COPOLYMER; Drop Point, Mettler: 143° C.) from Honeywell, A-C 597 (PROPYLENE-MALEIC ANHYDRIDE COPOLYMER; Drop Point, Mettler: 141° C.) from Honeywell, ZeMac® copolymers (from VERTELLUS) which are 1:1 copolymers of ethylene and maleic anhydride, polyisobutylene-maleic anhydride sold under the trade name ISOBAM (from Kuraray), polyisoprene-graft-maleic anhydride sold by Sigma Aldrich, poly(maleic anhydride-octadecene) sold by Chevron Philips Chemcial Co., poly (ethylene-co-butyl acrylate-co-maleic anhydride) sold under the trade name of Lotader (e.g. 2210, 3210, 4210, and 3410 grades) by Arkema, copolymers in which the butyl acrylate is replaced by other alkyl acrylates (including methyl acrylate [grades 3430, 4404, and 4503] and ethyl acrylate [grades 6200, 8200, 3300, TX 8030, 7500, 5500, 4700, and 4720) also sold by Arkema under the Lotader name, and isobutylene maleic anhydride copolymer sold under the name ACO-5013 by ISP.

According to other embodiments of the present invention, the polar modified polymer is not a wax. In accordance with these embodiments of the present invention, the polar modified polymer is based upon a homopolymer and/or copolymer of hydrophobic monomer(s) and has a weight-average molecular weight Mw of less than or equal to 1,000,000 g/mol, preferably of 1000 to 250,000 g/mol and particularly preferably of 5,000 to 50,000 g/mol, including all ranges and subranges therebetween.

In accordance with these embodiments, the polar modified polymer can be of any form typically associated with polymers such as, for example, block copolymer, a grafted copolymer or an alternating copolymer. For example, the polar modified polymer can contain a hydrophobic backbone (such as polypropylene and/or polyethylene) onto which hydrophilic groups (such as maleic anhydride) have been attached by any means including, for example, grafting. The attached groups can have any orienation (for example, atactic, isotactic or syndiotactic along the backbone).

Preferably, the oil soluble polar modified polymer(s) represent from about 1% to about 30% of the total weight of the composition, more preferably from about 2.5% to about 15% of the total weight of the composition, and most preferably from about 5% to about 10%, including all ranges and subranges therebetween.

Amine Compound

According to the present invention, compositions comprising at least one amine compound selected from the group consisting of a basic amino acid and a vinylamine/vinylamide copolymer are provided.

Basic Amino Acid

According to preferred embodiments of the present invention, compositions comprising at least one basic amino acid are provided. Exemplary basic amino acids include, but are not limited to, basic amino acids such as arginine, lysine, alanine, leucine, isoleucine, oxylysine and histidine, and derivatives and/or polymers thereof. A particularly preferred basic amino acid is arginine (available from Ajinomoto).

Preferably, the basic amino acid(s) represents from about 0.5% to about 7.5% of the total weight of the composition, more preferably from about 0.75% to about 5% of the total weight of the composition, and most preferably from about 1% to about 3%, including all ranges and subranges therebetween.

Vinylamine/Vinylamide Coplymer

According to preferred embodiments of the present invention, compositions comprising at least one vinylamine/vinylamide copolymer are provided. Suitable vinylamine/vinylamide copolymers are disclosed in U.S. patent application publication nos. 2008/0260666 and 2009/0269295, the entire contents of both of which are incorporated herein by reference.

According to preferred embodiments of the present invention, the at least one vinylamine/vinylamide copolymer may comprise from 10 to 60 mol % of units of formula A, such as from 20 to 40 mol %.

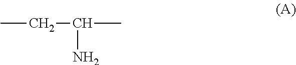

According to preferred embodiments of the present invention, the at least one vinylamine/vinylamide copolymer may comprise from 30 to 90 mol % of unit of formula B, such as from 60 to 80 mol %.

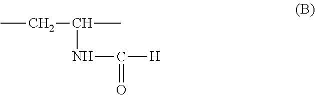

The copolymers disclosed herein can be obtained by partial hydrolysis of polyvinylformamide. The hydrolysis can be carried out in an acidic or basic medium.

The at least one vinylamine/vinylamide copolymer as disclosed herein, can optionally comprise at least one additional monomer unit. When present in the at least one vinylamine/vinylamide copolymer, the at least one additional monomer unit is preferably present in an amount less than 20 mol % of the copolymer.

According to one embodiment of the present invention, the at least one vinylamine/vinylamide copolymer is composed solely of units A and of units B.

Preferably, the weight-average molecular weight, measured by light diffraction, is from 10,000 to 30,000,000 g/mol, for example from 40,000 to 1,000,000 g/mol, such as from 100,000 to 500,000 g/mol, including all ranges and subranges therebetween.

Preferably, the cationic charge density of the at least one vinylamine/vinylamide copolymer at pH 5 is from 2 meq/g to 20 meq/g, for instance from 2.5 to 15 meq/g, such as from 3.5 to 10 meq/g, including all ranges and subranges therebetween.

Specific examples of suitable polymers include vinylamine/vinylamide copolymers sold under the name LUPAMIN 9030 and 9010 by BASF.

Preferably, the vinylamine/vinylamide copolymer (s) represents from about 1% to about 10% of the total weight of the composition, more preferably from about 1.5% to about 7.5% of the total weight of the composition, and most preferably from about 2% to about 5%, including all ranges and subranges therebetween.

Reaction Product

According to preferred embodiments of the present invention, the oil-soluble polar modified polymer is reacted with the at least one amine compound selected from the group consisting of a basic amino acid and a vinylamine/vinylamide copolymer to form an imine linkage between the compounds (for example, between the amine of the amine compound and maleic anhydride groups of the oil-soluble polar modified polymer). The resulting product provides an amphilic network capable of trapping liquid (for example, water) therein. Of course, the amount of basic amino acid present affects the amount of linkages which, in turn, affects the properties of the resulting product. For example, a loosely structure network containing fewer linkages may provide a more fluid product. A tighter network may provide a mascara with more volumization properties. A looser network allows for more water to be incorporated into the structure which, in turn, allows for more components typically found in aqueous phase (such as, for example, water-soluble or dispersible film forming agents) to be incorporated into the structure as well.

Aqueous Polyurethane Dispersion

The compositions of the present invention can also contain an aqueous polyurethane dispersion. Thus, according to preferred embodiments of the present invention, compositions further comprising at least one aqueous polyurethane dispersion are provided. “Aqueous polyurethane dispersion” as used herein means the aqueous polyurethane dispersions disclosed in U.S. Pat. No. 7,445,770 and/or U.S. Pat. No. 7,452,770, the entire contents of both of which are hereby incorporated by reference.

More specifically, the aqueous polyurethane dispersions of the present invention are preferably the reaction products of:

A) a prepolymer according to the formula:

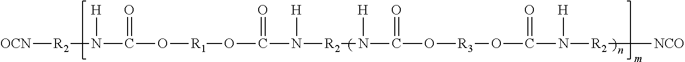

wherein R₁ represents a bivalent radical of a dihydroxyl functional compound, R₂ represents a hydrocarbon radical of an aliphatic or cycloaliphatic polyisocyanate, R₃ represents a radical of a low molecular weight diol, optionally substituted with ionic groups, n is from 0 to 5, and m is >1;

B) at least one chain extender according to the formula: H₂N—R₄—NH₂ wherein R₄ represents an alkylene or alkylene oxide radical not substituted with ionic or potentially ionic groups; and

C) at least one chain extender according to the formula: H₂N —R₅—NH₂ wherein R₅ represents an alkylene radical substituted with ionic or potentially ionic groups.

Suitable dihydroxyl compounds for providing the bivalent radical R₁ include those having two hydroxy groups and having number average molecular weights of from about 700 to about 16,000, and preferably from about 750 to about 5000. Examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. The polyester polyols, polyether polyols and polyhydroxy polycarbonates are preferred. Mixtures of various such compounds are also within the scope of the present invention.

Suitable polyisocyanates for providing the hydrocarbon radical R₂ include organic diisocyanates having a molecular weight of from about 112 to 1,000, and preferably from about 140 to 400. Preferred diisocyanates are those represented by the general formula R₂(NCO)₂ indicated above in which R₂ represents a divalent aliphatic hydrocarbon group having from 4 to 18 carbon atoms, a divalent cycloaliphatic hydrocarbon group having from 5 to 15 carbon atoms, a divalent araliphatic hydrocarbon group having from 7 to 15 carbon atoms or a divalent aromatic hydrocarbon group having 6-15 carbon atoms. Examples of the organic diisocyanates which are suitable include tetramethylene diisocyanate, 1,6-hexamethylene diisocyanate, dodecamethylene diisocyanate, cyclohexane-1,3- and -1,4-diisocyanate, 1-isocyanato-3-isocyanatomethyl-3,5,5-trimethylcyclohexane (isophorone diisocyanate or IPDI), bis-(4-isocyanatocyclohexyl)-methane, 1,3- and 1,4-bis(isocyanatomethyl)-cyclohexane, bis-(4-isocyanato-3-methyl-cyclohexyl)-methane, isomers of toluene diisocyanate (TDI) such as 2,4-diisocyanatotoluene, 2,6-diisocyanatotoluene, mixtures of these isomers, hydrogenated TDI, 4,4′-diisocyanato diphenyl methane and its isomeric mixtures with 2,4′- and optionally 2,2′-diisocyanato diphenylmethane, and 1,5-diisocyanato naphthalene. Mixtures of diisocyanates can, of course, be used. Preferred diisocyanates are aliphatic and cycloaliphatic diisocyanates. Particularly preferred are 1,6-hexamethylene diisocyanate and isophorone diisocyanate.

“Low molecular weight diols” in the context of R₃ means diols having a molecular weight from about 62 to 700, preferably 62 to 200. They may contain aliphatic, alicyclic or aromatic groups. Preferred compounds contain only aliphatic groups. The low molecular weight diols having up to about 20 carbon atoms per molecule include ethylene glycol, diethylene glycol, propane 1,2-diol, propane 1,3-diol, butane 1,4-diol, butylene 1,3-glycol, neopentyl glycol, butyl ethyl propane diol, cyclohexane diol, 1,4-cyclohexane dimethanol, hexane 1,6-diol, bisphenol A (2,2-bis(4-hydroxyphenyl)propane), hydrogenated bisphenol A (2,2-bis(4-hydroxycyclohexyl)propane), and mixtures thereof. Optionally, the low molecular weight diols may contain ionic or potentially ionic groups. Suitable lower molecular weight diols containing ionic or potentially ionic groups are those disclosed in U.S. Pat. No. 3,412,054, the contents of which is hereby incorporated by reference. Preferred compounds include dimethylol butanoic acid (DMBA), dimethylol propionic acid (DMBA) and carboxyl-containing caprolactone polyester diol. If lower molecular weight diols containing ionic or potentially ionic groups are used, they are preferably used in an amount such that <0.30 meq of COOH per gram of polyurethane in the polyurethane dispersion are present.

The prepolymer is chain extended using two classes of chain extenders. First, compounds having the formula: H₂N —R₄—NH₂ wherein R₄ represents an alkylene or alkylene oxide radical not substituted with ionic or potentially ionic groups. Alkylene diamines include hydrazine, ethylenediamine, propylenediamine, 1,4-butylenediamine and piperazine. The alkylene oxide diamines include 3-{2-[2-(3-aminopropoxy)ethoxy]ethoxy}propylamine (also known as dipropylamine diethyleneglycol or DPA-DEG available from Tomah Products, Milton, Wis.), 2-methyl-1,5-pentanediamine (Dytec A from DuPont), hexane diamine, isophorone diamine, and 4,4-methylenedi-(cyclohexylamine), and the DPA-series ether amines available from Tomah Products, Milton, Wis., including dipropylamine propyleneglycol, dipropylamine dipropyleneglycol, dipropylamine tripropyleneglycol, dipropylamine poly(propylene glycol), dipropylamine ethyleneglycol, dipropylamine poly(ethylene glycol), dipropylamine 1,3-propane diol, dipropylamine 2-methyl-1,3-propane diol, dipropylamine 1,4-butane diol, dipropylamine 1,3-butane diol, dipropylamine 1,6-hexane diol and dipropylamine cyclohexane-1,4-dimethanol. Mixtures of the listed diamines may also be used.

The second class of chain extenders are compounds having the formula: H₂N—R₅—NH₂ wherein R₅ represents an alkylene radical substituted with ionic or potentially ionic groups. Such compounds have an ionic or potentially ionic group and two groups that are reactive with isocyanate groups. Such compounds contain two isocyanate-reactive groups and an ionic group or group capable of forming an ionic group. The ionic group or potentially ionic group can be selected from the group consisting of ternary or quaternary ammonium groups, groups convertible into such a group, a carboxyl group, a carboxylate group, a sulfonic acid group and a sulfonate group. The at least partial conversion of the groups convertible into salt groups of the type mentioned may take place before or during the mixing with water. Specific compounds include diaminosulfonates, such as for example the sodium salt of N-(2-aminoethyl)-2-aminoethane sulfonic acid (AAS) or the sodium salt of N-(2-aminoethyl)-2-aminopropionic acid.

The polyurethane according to the invention may also include compounds which are situated in each case at the chain ends and terminate said chains (chain terminators) as described in U.S. Pat. No. 7,445,770 and/or U.S. Pat. No. 7,452,770.

Preferably, the aqueous polyurethane dispersion has a viscosity of less than 2000 mPa·s at 23 □C, preferably less than 1500, preferably less than 1000, including all ranges and subranges therebetween. Further preferably, the aqueous polyurethane dispersion has a glass transition temperature below 0° C.

Also preferably, the aqueous polyurethane dispersion has a solids content based on the weigh of the dispersion of from 20% to 60%, preferably from 25% to 55% and preferably from 30% to 50%, including all ranges and subranges therebetween.

Suitiable aqueous polyurethane dispersions for use in the present invention include, but are not limited to, aqueous polyurethane dispersions sold under the BAYCUSAN® name by Bayer such as, for example, BAYCUSAN® C1000 (polyurethane-34), BAYCUSAN® C1001 (polyurethane-34), BAYCUSAN® C1003 (polyurethane-32), and BAYCUSAN® C1004 (polyurethane-35).

According to preferred embodiments, the at least one aqueous polyurethane dispersion is present in the composition of the present invention in an amount ranging from about 5 to 50% by weight (non-dry weight basis), more preferably from about 10 to about 45% by weight, more preferably from about 20 to about 40% by weight based on the total weight of the composition, including all ranges and subranges within these ranges.

Water

The composition of the present invention can also contain water. The water, if present, is typically present in an amount of from about 5% to about 50% by weight, such as from about 10% to about 40% by weight, such as from about 25% to about 35% by weight, including all ranges and subranges therebetween, all weights being based on the total weight of the composition. According to particularly preferred embodiments, sufficient water is present to form a water-in-oil emulsion.

Optional Ingredients

Non-Volatile Oil for Oil-Soluble Polar Modified Polymer

The cosmetic composition of the present invention can comprise at least one non-volatile oil capable of dissolving the oil-soluble polar modified polymer. As used herein, the term “non-volatile” means having a boiling point of greater than about 100 degrees C.

Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to 0₂₆ fatty alcohols, for instance oleyl alcohol; and
mixtures thereof.

The at least one non-volatile oil, if present, is preferably present in the cosmetic composition of the invention in an amount of from about 0.5% to about 15% by weight, such as from about 1% to about 10% by weight, such as from about 2% to about 5% by weight, all weights based on the total weight of the composition.

Volatile Solvent

The cosmetic composition of the present invention can comprise at least one volatile solvent. In an embodiment of the present invention, at least one volatile solvent may be present and be chosen from a volatile silicone oil or a volatile non-silicone oil.

Suitable volatile silicone oils include, but are not limited to, linear or cyclic silicone oils having a viscosity at room temperature less than or equal to 6 cSt and having from 2 to 7 silicon atoms, these silicones being optionally substituted with alkyl or alkoxy groups of 1 to carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | | Viscosity |
|---|---|---|
| Compound | Flash Point (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (3St) from Dow Corning | 102 | 3 |

Suitable volatile non-silicone oils may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

Non-limiting examples of volatile non-silicone oils are listed in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C11-C13) | 62 |
| Isopar H (isoparaffin C11-C12) | 56 |

In general, the at least one volatile solvent, if present, is preferably present in the composition in an amount of from about 5% to about 80% by weight, such as from about 10% to about 60% by weight, and from about 20% to about 40% by weight, all weights based on the total weight of the composition.

Compositions of the present invention can optionally further comprise any additive usually used in the field(s) under consideration. For example, dispersants such as poly(12-hydroxystearic acid), antioxidants, essential oils, sunscreens, preserving agents, fragrances, fillers, neutralizing agents, cosmetic and dermatological active agents such as, for example, emollients, moisturizers, vitamins, essential fatty acids, surfactants, silicone elastomers, pasty compounds, viscosity increasing agents such as waxes or liposoluble/lipodispersible polymers, and mixtures thereof can be added. A non-exhaustive listing of such ingredients can be found in U.S. patent application publication no. 2004/0170586, the entire contents of which are hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

One particularly preferred embodiment of the present invention is an emulsion which is substantially free of surfactant (that is, less than 3% of surfactant), essentially free of surfactant (that is, less than 2% surfactant), or free of surfactant (that is, less than 0.5% surfactant).

Another particularly preferred embodiment of the present invention is a composition which contains so little elastomer that the presence of such elastomer not affect the cosmetic properties of the composition. Preferably, the compositions are substantially free of such elastomers (i.e., contain less than about 0.5% elastomer), essentially free of such elastomers (i.e., contain less than about 0.25% elastomer) or free of such elastomer (i.e., contain no elastomer).

According to other preferred embodiments, methods of treating, caring for and/or enhancing the appearance of keratinous material by applying compositions of the present invention to the keratinous material in an amount sufficient to treat, care for and/or enhance the appearance of the keratinous material are provided. In accordance with these preceding preferred embodiments, the compositions of the present invention are applied topically to the desired area of the keratin material in an amount sufficient to treat, care for and/or enhance the appearance of the keratinous material. The compositions may be applied to the desired area as needed, preferably once or twice daily, more preferably once daily and then preferably allowed to dry before subjecting to contact such as with clothing or other objects (for example, a glass or a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to a preferred embodiment of the present invention, compositions having improved cosmetic properties such as, for example, improved waterproof characteristics, improved feel upon application (for example, texture, reduced drag or tackiness), increased anti-smudging properties, shine/color characteristics and/or increased long wear properties are provided.

According to other embodiments of the present invention, methods of improving the anti-smudging, waterproof, transfer-resistance and/or long wear properties of a composition and/or removability from a tube or container, comprising adding at least one basic amino acid and at least one oil-soluble polar modified polymer to the composition are provided.

It has surprisingly been discovered that the composition of the present invention forms a long wearing, water-resistant eye makeup composition having a unique texture and feel and enhanced shine. Without intending to be bound by theory, it is believed that the combination of oil-soluble polar modified polymer and at least one basic amino acid forms a product that is able to entrap large amounts of solvent (for example, water) molecules within its matrix. Consequently, the composition is water-resistant and long-wear without the need for having to employ conventional film forming polymers. Moreover, the product provides enhanced shine and/or volume.

According to other embodiments of the present invention, methods of improving the anti-smudging, waterproof, transfer-resistance and/or long wear properties of a composition, comprising adding at least one basic amino acid and at least one oil-soluble polar modified polymer to the composition are provided.

It has surprisingly been discovered that the composition of the present invention forms a long wearing, water-resistant eye makeup composition having a unique texture and feel and enhanced shine. Without intending to be bound by theory, it is believed that the combination of oil-soluble polar modified polymer and at least one basic amino acid forms a product that is able to entrap large amounts of solvent (for example, water) molecules within its matrix. Consequently, the composition is water-resistant and long-wear without the need for having to employ conventional film forming polymers. Moreover, the product provides enhanced shine and/or volume.

According to other embodiments of the present invention, methods of improving the anti-smudging, waterproof, transfer-resistance, volumization and/or long wear properties of a composition, comprising adding at least one vinylamine/vinylamide copolymer and at least one oil-soluble polar modified polymer to the composition are provided.

It has surprisingly been discovered that the composition of the present invention forms a long wearing, water-resistant eye makeup composition having a unique texture and feel and enhanced shine. Without intending to be bound by theory, it is believed that the combination of oil-soluble polar modified polymer and at least one vinylamine/vinylamide copolymer forms a product that is able to entrap large amounts of solvent (for example, water) molecules within its matrix. Consequently, the composition is water-resistant and long-wear without the need for having to employ conventional film forming polymers. Moreover, the product provides enhanced shine and/or volume.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLE 1

Mascara Composition 100.000 bal %:

| Phase | Chemical Name | % wt/wt |
|---|---|---|
| A | Caprylic/capric Triglyceride | 1.00% |
| A | PPMA | 13.33% |
| A | Isododecane | 5.00% |
| A | Ethylparaben | 0.20% |
| B | DI Water | 34.42% |
| B | Disodium EDTA | 0.10% |
| B | Methylparaben | 0.35% |
| B | Pentylene Glycol | 2.00% |
| B | C20-40 Pareth-95 (and) Polyethylene | 2.00% |
| B | Arginnee | 1.00% |
| C | Pigment Dispersions | 10.00% |
| C | Simethicone | 0.10% |
| D | AQUEOUS POLYURETHANE POLYESTER | 30.00% |
| | DISPERSION | |
| E | PHENOXYETHANOL | 0.50% |

### EXAMPLE 2

| Phase | Chemical Name | % wt/wt |
|---|---|---|
| A | Caprylic/capric Triglyceride | 1.00 |
| A | Polylene ethylene MALEIC ANHYDRIDE | 9.33 |
| | COPOLYMER (PPMA) | |
| A | Iron Oxides | 8.00 |
| A | Isododecane | 40.42 |
| A | Ethylparaben | 0.20 |
| B | DI Water | 35.00 |
| B | Disodium EDTA | 0.10 |
| B | Potassium Cetyl Phosphate | 2.00 |
| B | Methylparaben | 0.35 |
| B | Pentylene Glycol | 2.00 |
| B | PVP | 0.00 |
| B | Arginne | 1.00 |
| C | Simethicone | 0.10 |
| C | PHENOXYETHANOL | 0.50 |
| Total | | 100 |

### EXAMPLE 3

| Phase | Chemical Name | % wt/wt |
|---|---|---|
| A | Caprylic/capric Triglyceride | 1.00 |
| A | Polylene ethylene MALEIC ANHYDRIDE | 9.33 |
| | COPOLYMER (PPMA) | |
| A | Iron Oxides | 8.00 |
| A | Isododecane | 40.42 |
| A | Ethylparaben | 0.20 |
| B | DI Water | 35.00 |
| B | Disodium EDTA | 0.10 |
| B | Potassium Cetyl Phosphate | 2.00 |
| B | Methylparaben | 0.35 |
| B | Pentylene Glycol | 2.00 |
| B | PVP | 0.00 |
| B | Lysine | 1.00 |
| C | Simethicone | 0.10 |
| C | PHENOXYETHANOL | 0.50 |
| Total | | 100 |

What is claimed is:

1. A mascara composition comprising:
(a) at least one oil-soluble polar modified polymer;
(b) at least one amine compound selected from the group consisting of a basic amino acid and a vinylamine/vinylamide copolymer.

2. The composition of claim 1, wherein the amine compound is a basic amino acid.

3. The composition of claim 1, wherein the amine compound is a vinylamine/vinylamide copolymer.

4. The composition of claim 1, further comprising water.

5. The composition of claim 2, further comprising at least one aqueous polyurethane dispersion.

6. The composition of claim 1, wherein the oil-soluble polar modified polymer is present in an amount of from about 1% to about 30% by weight, based on the weight of the composition.

7. The composition of claim 1, wherein the oil-soluble polar modified polymer is a polypropylene and/or polyethylene-maleic anhydride modified wax.

8. The composition of claim 2, wherein the basic amino acid is lysine and/or arginine.

9. The composition of claim 2, wherein the basic amino acid is arginine.

10. The composition of claim 2, wherein the basic amino acid is present in an amount of from about 0.5% to about 7.5% by weight based on the weight of the composition.

11. The composition of claim 3, wherein the vinylamine/vinylamide copolymer comprises from 10 to 60 mol % of units of formula A:

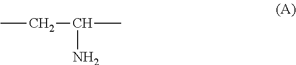

12. The composition of claim 3, wherein the vinylamine/vinylamide copolymer comprises from 30 to 90 mol % of unit of formula B:

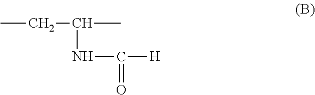

13. The composition of claim 3, wherein the vinylamine/vinylamide copolymer is composed solely of units A and of units B identified below:

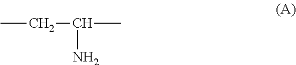

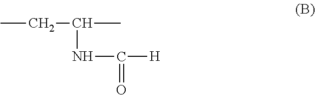

14. The composition of claim 3, wherein the vinylamine/vinylamide copolymer is present in an amount of from about 1% to about 10% by weight based on the weight of the composition.

15. The composition of claim 4, wherein water is present in an amount of from about 5% to about 50% by weight, based on the weight of the composition.

16. The composition of claim 5, wherein the aqueous polyurethane dispersion is present in an amount from about 5% to about 50% of the total weight of the composition.

17. The composition of claim 5, wherein the aqueous polyurethane dispersion comprises polyurethane-35.

18. The composition of claim 2, wherein the oil-soluble polar modified polymer and the basic amino acid form a reaction product.

19. The composition of claim 3, wherein the oil-soluble polar modified polymer and the vinylamine/vinylamide copolymer form a reaction product.

20. A method of making up eyelashes comprising applying the composition of claim 1 to eyelashes.
